(19) United States
(12) Patent Application Publication
(10) Pub. No.: US 2011/0160669 A1
(43) Pub. Date: Jun. 30, 2011

(54) Inserter for Transcutaneous Device

(76) Inventors: Steffen Gyrn, Ringsted (DK); Frederik Hjort, Dianalund (DK)

(21) Appl. No.: 13/000,464

(22) PCT Filed: Jul. 3, 2009
(86) PCT No.: PCT/EP2009/058377

§ 371 Date: Feb. 14, 2011

Related U.S. Application Data
(60) Provisional application No. 61/134,173, filed on Jul. 7, 2008.

(51) Int. Cl. A61M 5/158; A61M 5/142

(52) U.S. Cl. 604/151; 604/164.12

(57) ABSTRACT

The present invention relates to a device comprising a transcutaneous device and an inserter which transcutaneous device has a mounting surface. The device is upon delivery arranged in a sterile packing which packing functions as inserter and is attached to the transcutanous device i.e. the packing and the inserter is one integrated unit. A device according to the present invention comprises a transcutaneous device (1) and an inserter (2). The transcutaneous device comprises—a mounting part (13) having a mounting surface (14),—a soft cannula (6)—a fluid path leading fluid from a fluid source to the soft cannula (6),—means for releasably attaching the transcutaneous device (1) to the inserter (2). The inserter (2) comprises—a cover providing an at least partly protection against the surroundings for the distal side of the transcutaneous device (1) at least before insertion of the soft cannula (6),—means for releasably attaching the inserter (2) to the transcutaneous device (1),—an insertion needle (5),—at least one handle (3, 4) for loading and releasing of the insertion needle (5),—at least one elastic member (19, 22) which is loaded upon activation of the at least one handle (3, 4) and afterwards released.

### TECHNICAL FIELD

The present invention relates to a device comprising a transcutaneous device and an inserter which transcutaneous device has a mounting surface. The device is upon delivery arranged in a sterile packing which packing functions as inserter and is attached to the transcutaneous device i.e. the packing and the inserter is one integrated unit.

### PRIOR ART

WO 2007/141210 discloses an assembly comprising a skin mountable device and a packing for this device. The packing of this device can be attached to the enclosed skin mountable device and then the packing can be used as an applicator for mounting the skin mountable device on the skin surface of the patient. When the packing is pushed in the direction of the housing i.e. along the skin surface, the packing will become detached from the skin mountable device. A body portion comprises a housing portion in which a cannula inserting mechanism is arranged i.e. the cannula inserting mechanism is not part of the packing and the cannula inserting mechanism according to this document is complex and comprises a coil-formed tension spring.

### SUMMARY OF THE INVENTION

The present invention relates to a device comprising a transcutaneous device and an inserter.

The transcutaneous device comprises
a mounting part having a mounting surface,
a soft cannula,
a fluid path leading fluid from a fluid source to the soft cannula,
means for releasably attaching the transcutaneous device to the inserter

The inserter comprises or is constituted of
a cover providing an at least partly protection against the surroundings for the distal side of the transcutaneous device at least before insertion of the soft cannula,
means for releasably attaching the inserter to the transcutaneous device,
an insertion needle,
at least one handle for loading and releasing of the insertion needle,
at least one elastic member which is loaded upon activation of the at least one handle and afterwards released.

According to one embodiment the device comprises two elastic members, a first elastic member can force the insertion needle into the transcutaneous position when released and a second elastic member can retract the insertion needle from the transcutaneous position when released.

According to one embodiment the at least one handle comprises two handles where a first handle is used to load a first elastic member and a second handle is used to release the same first elastic member.

According to one embodiment the protective cover is made of a hard material having openings for one or more handles.

According to one embodiment the cover has an opening adapted for receiving a delivery unit comprising a pump, a reservoir for fluid and a controller in order to attach the delivery unit to the transcutaneous device.

According to one embodiment the inserter can comprise a sliding part connected to the first handle and a contact part connected to the second handle, further the sliding part is connected to the contact part via the first elastic member. The second handle can release a locking part preventing the contact part from moving towards the sliding part. Further the first elastic member can consist of a rubber band or another spring which is loaded upon expansion. The contact part can consist of a part having a contact surface pushing against a surface of a needle holder holding the insertion needle.

According to one embodiment the soft cannula of the transcutaneous device is attached to a cannula part which cannula part is connected fluid tight to the reservoir after insertion. The fluid tight connection to the reservoir can be obtained after insertion as either the cannula part or a cannula holder or both is/are provided with a gasket or the fluid tight connection to the reservoir can be obtained both before and after insertion as the cannula part is connected to a stationary connector part with a flexible or movable connection. If the fluid tight connection is provided with a flexible or movable connection then the movable connection might be pivoted relative to the mounting part. This flexible connection can be constituted by a soft tube connected to the inlet of the cannula part and to the outlet of the connector part.

According to one embodiment the transcutaneous device comprises longitudinal rails corresponding to grooves or hooks on the inserter.

According to one embodiment the fluid source can be a reservoir attached to the mounting part.

According to one embodiment the reservoir can contain a medication such as insulin.

According to one embodiment the insertion point for the cannula can be separated from the main part of the mounting part by a flexible band of a compliant material.

### DETAILED DESCRIPTION OF THE INVENTION

Embodiments of the invention will now be described with reference to the figures in which:

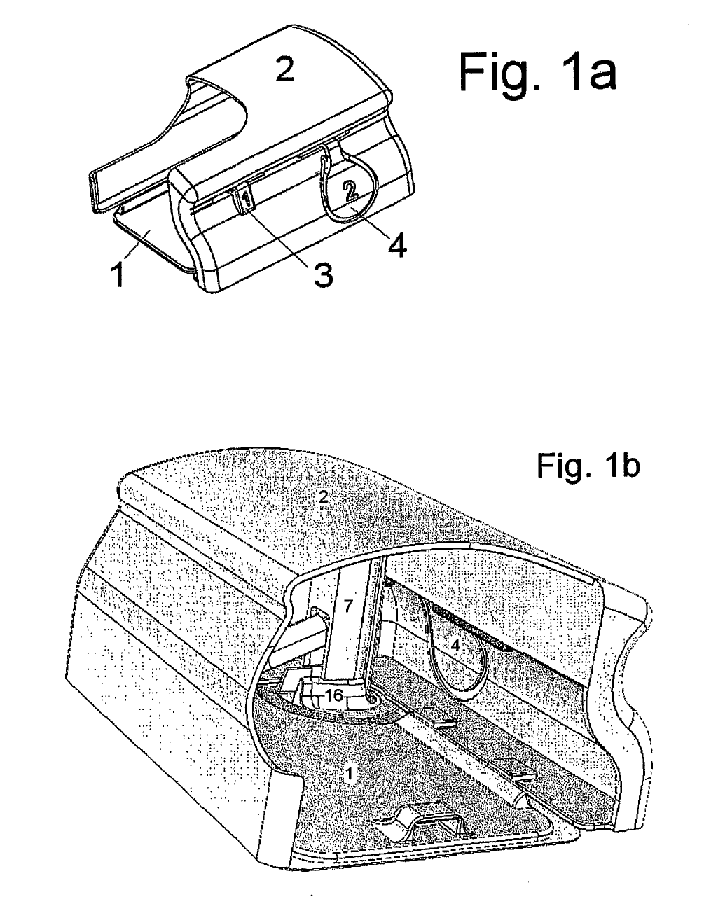

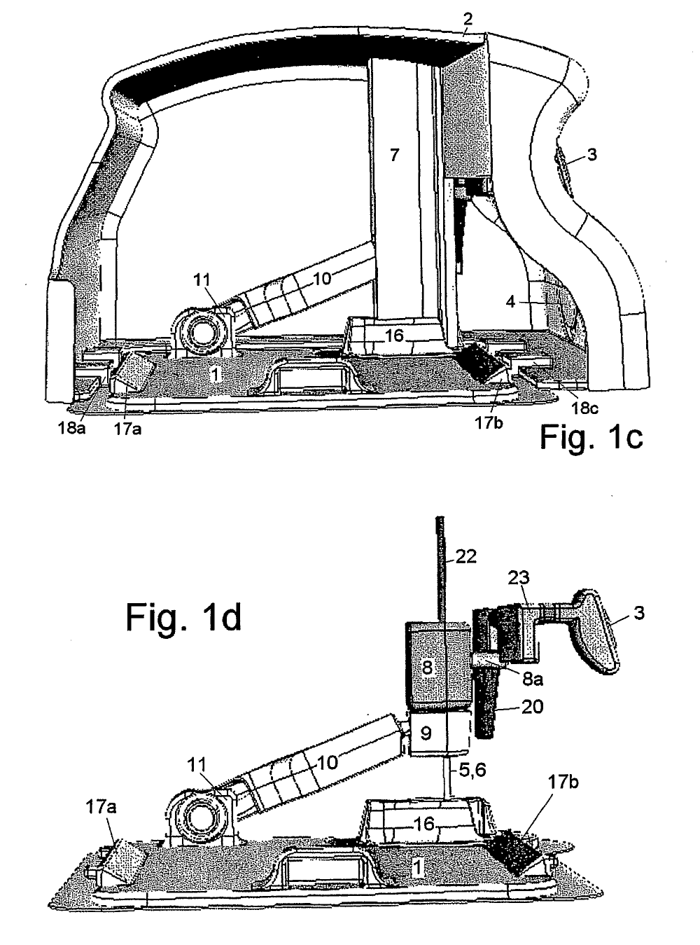

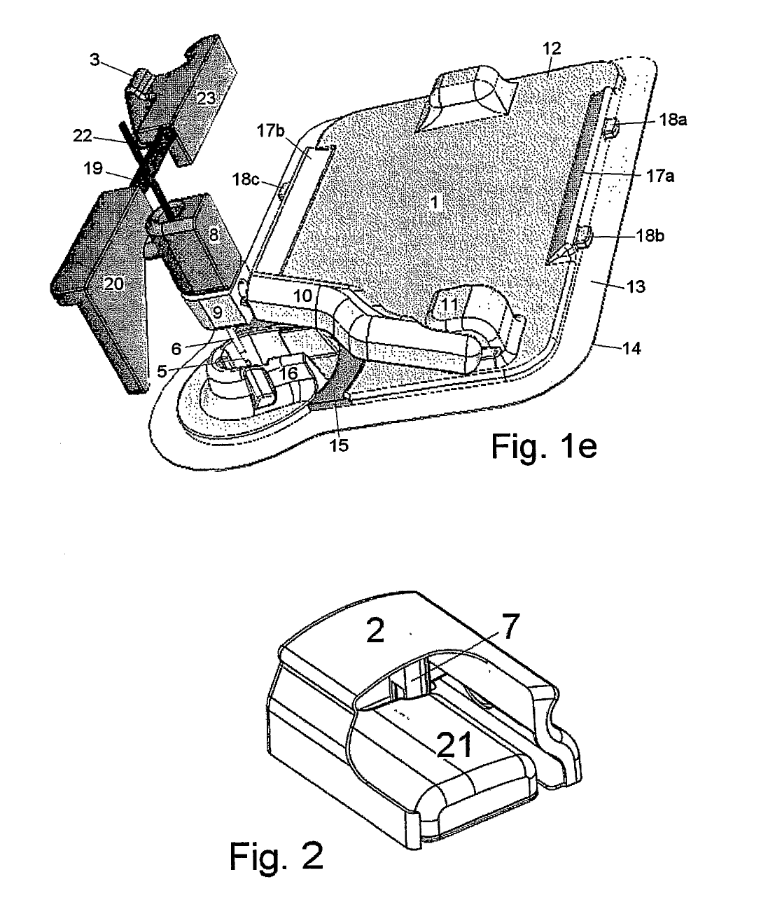

FIG. 1a, 1b and 1c show an embodiment of an assembled device seen from three different angles, and 1d and 1e show the same embodiment without the hard top inserter from two different angles.

FIG. 2 shows the same embodiment as FIG. 1a-1e having a delivery device comprising at least a reservoir in working position.

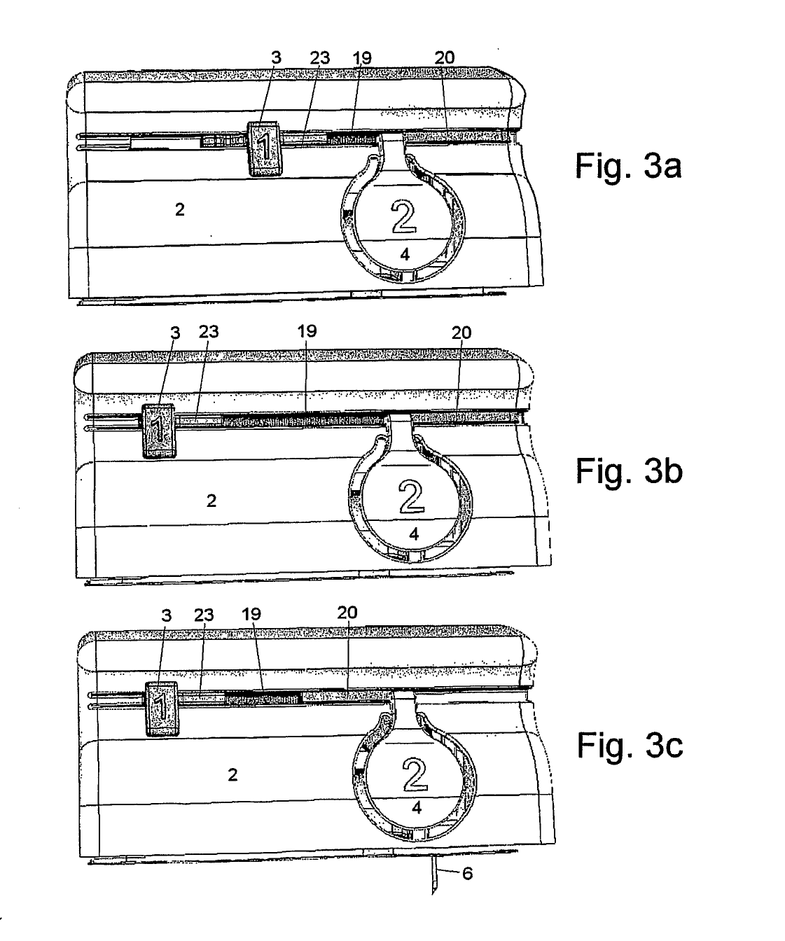

FIGS. 3a, 3b and 3c show the positions that the activating handle and the contact part takes during insertion.

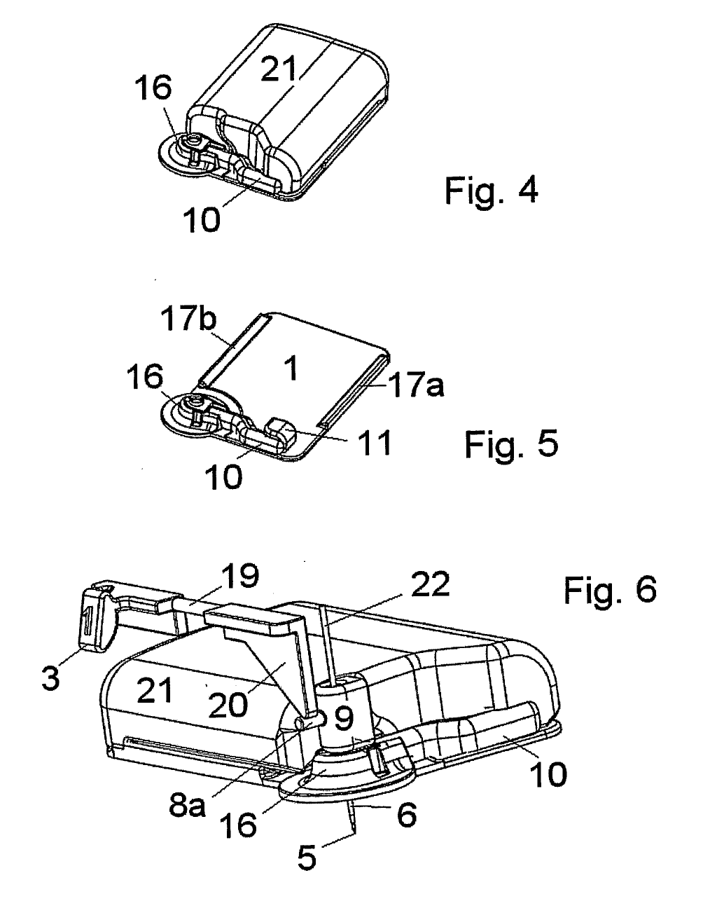

FIG. 4 shows a transcutaneous device after it has been positioned on the patients skin where the delivery device is in working position and the packing/inserter has been removed.

FIG. 5 shows a transcutaneous device after it has been positioned on the patients skin without both the delivery device and the packing/inserter.

FIG. 6 shows the position of the sliding part, the contact part, the first elastic member and the cannula parts just after insertion and before removal of the packing/inserter.

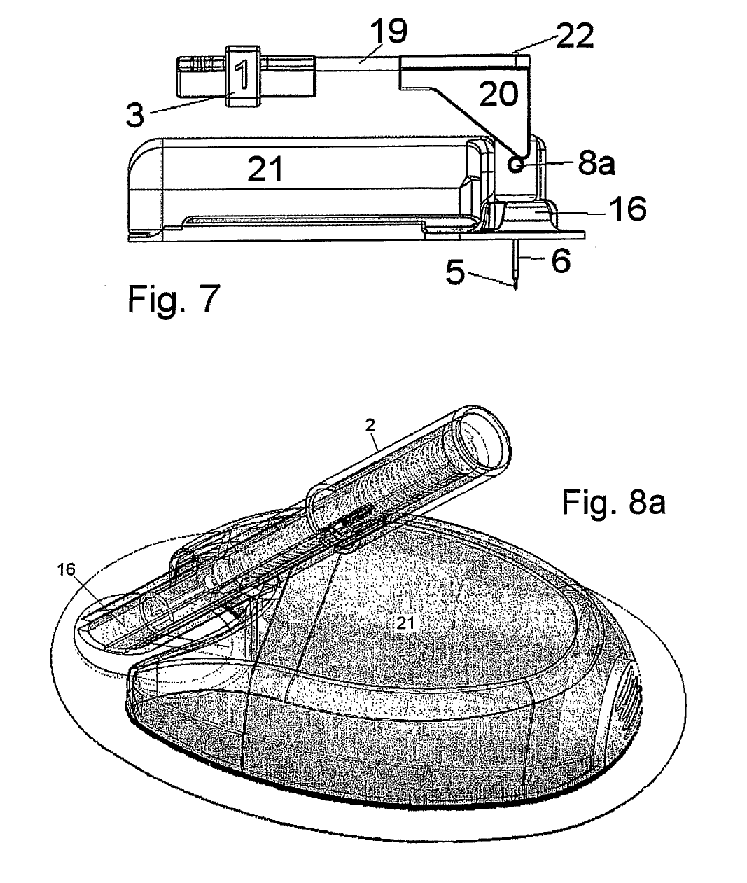

FIG. 7 shows the same parts as in FIG. 6 seen from the side.

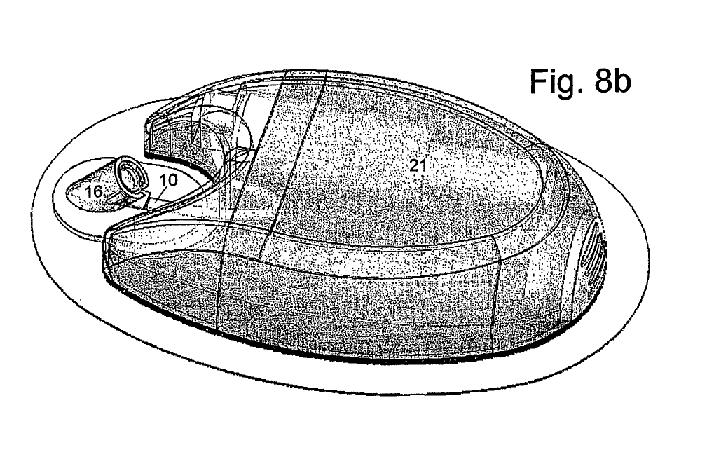

FIGS. 8a and 8b show another embodiment of a transcutaneous device combined with an inserter.

FIG. 1a shows a device according to the invention where one end of the transcutaneous device 1 can be seen and the outer surface of an inserter 2 is provided with a first handle 3 for loading and a second handle 4 for releasing of the insertion needle 5, upon release the insertion needle 5 in combination with a soft cannula 6 is inserted transcutaneously into a patients skin.

FIG. 1b shows a view inside the embodiment of FIG. 1a. From this angle it is possible to see internal guiding means 7 of the inserter which guiding means 7 before insertion hold the insertion needle holder 8 and the cannula part 9. The needle holder 8 and the cannula part 9 can slide down inside the guiding means 7. The cannula part 9 is provided with a soft cannula 6 and before and during insertion the insertion needle 5 is normally placed inside the hollow cannula 6.

FIG. 1c, 1d and 1e show the same embodiment as FIGS. 1a and 1b from an angle showing a connector arm 10 providing a fluid path from a connector part 11 to the cannula part 9. A tube of a soft and compliant material can be placed inside the connector arm 10 and provide a movable coupling to the cannula part 9. The connector part 11 is further provided with an access to a fluid source e.g. in the form of a closed reservoir placed inside on the upper surface of the transcutaneous device.

FIG. 1e and 1d also show two elastic members, a first member 19 and a second member 22. The first member 19 is loaded when the first handle 3 and a sliding member 22 which is attached to this handle are pulled away from the contact part 20. The second member 22 is loaded when the insertion holding member 8 is forced down by an inclined surface of the contact member 20.

The transcutaneous device 1 according to FIG. 1a-1e comprises a relatively hard plate 12 placed on a mounting part 13 which mounting part 13 is provided with a mounting surface 14. The mounting surface 14 can be attached to the patient and normally the mounting surface is provided with an adhesive. The connector part 11 is fastened unreleasably to the mounting part 12 and the connecting arm 10 can pivot relative to the connector part 11. The transcutaneous device 1 is provided with means 17a, 17b for joining the delivery device to the transcutaneous device and with means 18a, 18b and 18c for attaching the inserter 2 to the transcutaneous device. The inserter 2 is normally released after insertion of the cannula 6 in order for the transcutaneous device to be as small as possible.

FIG. 2 shows the same embodiment as in FIG. 1a-1e but in FIG. 2 a delivery device has been inserted into its working position i.e. a fluid path has been formed between the not shown reservoir in the delivery device and the cannula. In this working position a small automatic pump will normally administer the medication to the patient for up to 3-7 days. As it is possible to remove and re-insert the delivery device it is also possible to take the delivery device out and refill or change the not shown reservoir.

FIGS. 3a, 3b and 3c illustrates the steps the inserter 2 passes during insertion of the cannula part 9.

FIG. 3a shows the inserter 2 in a state before insertion. The first handle 3 and the thereto attached sliding part 23 are in a position relatively close to the contact part 20. The first elastic member 19 is in an unloaded state and the soft cannula 6 and the insertion needle 5 are not visible as they are still hidden inside the inserter 2.

FIG. 3b also shows the inserter 2 in a state before insertion. The first handle 3 and the thereto attached sliding part 23 are in a position as far away from the contact part 20 as possible. The first elastic member 19 is in a loaded state, the soft cannula 6 and the insertion needle 5 are still in the start position and not visible. The distance between the sliding part 23 and the contact member 20 can only be maintained because a part of the second handle 4 prevents the contact part 20 from moving toward the sliding part 23.

FIG. 3c shows the inserter 2 in a state after insertion of the soft cannula. The first handle 3 and the sliding part 23 are in the same position as in FIG. 3b but as the user pushed the second handle 4 toward the centre of the inserter 2, the contact member 20 will move toward the sliding part 23, this first forces the needle holder 9 down causing the insertion needle 5 and the soft cannula 6 to be inserted transcutaneously as the inclined surface of the contact part 20 is pushing down on the protruding member 8a. The protruding member 8a is attached to the needle holder 9. When the inclined surface of the contact part 8 has passed the protruding member 8a, then the second elastic member is loaded and the needle holder 9 is free to return to its start position. The contact between the contact part 20 and the protruding member 8a is made smooth and predictable as the end corner of the inclined surface is rounded.

FIG. 4 shows the transcutaneous device 1 after removal of the inserter 2 having a delivery device 21 in working position.

FIG. 5 shows the transcutaneous device 1 after removal of the inserter 2 without a delivery device 21 attached thereto.

FIGS. 6 and 7 show the transcutaneous device 1 combined with a delivery device 21. Further the parts of the inserter which are not a part of the protective cover are shown. In FIGS. 6 and 7 the inserter parts are shown in a non-stable state where the contact member 20 has forced the needle holder 9 fully down, the cannula part 8 is hidden and locked inside a cannula holder 16 which cannula holder 16 provides a close fit to the cannula part 8.

FIGS. 8a and 8b show another embodiment of a transcutaneous device combined with an inserter. This inserter though is pencil-shaped and pre-loaded upon delivery having automatic insertion when activated by the user and automatic retraction, i.e. the retraction is not user activated.

The cannula holder 16 is inclined in the transcutaneous device shown in this embodiment.

1. A device comprising:
a transcutaneous device comprising:
a mounting part having a mounting surface,
a soft cannula
a fluid path leading fluid from a fluid source to the soft cannula, and
an inserter comprising:
a cover at least partly providing protection against the surroundings for a distal side of the transcutaneous device at least before insertion of the soft cannula,
means an attachment member for releasably attaching the inserter to the transcutaneous device,
an insertion needle,
at least one handle for loading and releasing of the insertion needle,
at least one elastic member which is loaded upon activation of the at least one handle and afterwards released and
wherein the transcutaneous device comprises an attachment member for releasably attaching the transcutaneous device to the inserter.

2. The device according to claim 1, wherein the device comprises two elastic members, a first elastic member that can force the insertion needle into the transcutaneous position when released and a second elastic member that can retract the insertion needle from the transcutaneous position when released.

3. The device according to claim 1, wherein the at least one handle comprises two handles where a first handle is used to load a first elastic member and a second handle is used to release the same first elastic member.

4. The device according to claim 1, wherein the protective cover is made of a hard material having openings for one or more handles.

5. The device according to claim 1, wherein the cover has an opening adapted for receiving a delivery unit comprising a pump, a reservoir for fluid and a controller in order to attach the delivery unit to the transcutaneous device.

6. The device according to claim 3, wherein the inserter comprises a sliding part connected to the first handle and a contact part connected to the second handle, further the sliding part is connected to the contact part via the first elastic member.

7. The device according to claim 6, wherein the second handle releases a locking part preventing the contact part from moving towards the sliding part.

8. The device according to claim 6, wherein the first elastic member consists of a rubber band or another spring which is loaded upon expansion.

9. The device according to claim 6, wherein contact part comprises a part having a contact surface pushing against a surface of a needle holder holding the insertion needle.

10. The device according to claim 1, wherein the soft cannula of the transcutaneous device is attached to a cannula part and the cannula part is connected to the reservoir after insertion in a fluid tight connection.

11. The device according to claim 10, wherein the fluid tight connection to the reservoir is obtained after insertion, the cannula part or a cannula holder or both is/are provided with a gasket.

12. The device according to claim 10, wherein the cannula part is connected to a stationary connector part with a flexible or movable connection so that the fluid tight connection to the reservoir is obtained both before and after insertion.

13. The device according to claim 12, wherein the movable connection can be pivoted relative to the mounting part.

14. The device according to claim 12, wherein the flexible connection comprises a soft tube connected to the inlet of the cannula part and to the outlet of the connector part.

15. The device according to claim 1, wherein the transcutaneous device comprises longitudinal rails corresponding to grooves or hooks on the inserter.

16. The device according to claim 1, wherein the fluid source is a reservoir attached to the mounting part.

17. The device according to claim 16, wherein the reservoir contains a medication.

18. The device according to claim 1, wherein an insertion point for the cannula is separated from a main part of the mounting part by a flexible band of a compliant material.

19. The device according to claim 17, wherein the medication comprises insulin.
